(19) United States
(12) Patent Application Publication
(10) Pub. No.: US 2012/0078223 A1
(43) Pub. Date: Mar. 29, 2012

(54) WARMING GARMENT AND METHOD FOR PROMOTING VASODILATION

(76) Inventor: Joan E. Spiegel, Natick, MA (US)

(21) Appl. No.: 12/957,089

(22) Filed: Nov. 30, 2010

Related U.S. Application Data
(60) Provisional application No. 61/386,504, filed on Sep. 25, 2010.

(51) Int. Cl. A61F 7/08; A61M 5/31; F24J 1/00; A41D 13/005; A61F 7/00

(52) U.S. Cl. 604/506; 2/69; 126/204; 126/263.01; 604/113

(57) ABSTRACT

A warming garment and method of making and using are provided. The warming garment may have an inner lining and an outer lining which may form an annular space in which an exothermic composition that is activated upon exposure to oxygen may be disposed. When worn, the warming garment may aid in inducing vasodilation upon a subject's limb through the emanation of heat from the exothermic composition. Once vasodilation is achieved, venipuncture or catheter insertion procedures may be properly initiated.

### RELATED APPLICATION

This application claims benefit to a U.S. Provisional Application No. 61/386,504 filed on Sep. 25, 2010 and which is incorporated by reference herein.

### BACKGROUND OF THE INVENTION

1. Field of the Invention

The present application relates to a warming garment and a method for venipuncture, in particular, a warming garment to aid in intravenous access and methods of making and using the same.

2. Description of Related Art

Venipuncture is a common medical procedure that is often practiced in patient care environments such as hospitals, clinics and various medical offices. Medical practitioners often administer this procedure to draw blood, provide intravenous feeding, or administer medication. The procedure is typically performed by securing a tourniquet around a patient's limb to constrict the flow of blood through the veins and causing a dilation of the veins known as vasodilation. Once vasodilation is achieved, the vein is located and punctured with a needle or catheter, a process known as venipuncture.

Achieving vasodilation in preparation for venipuncture or intravenous catheter insertion can often be difficult for medical practitioners. Such difficulties can be caused by a patient's fear of pain, cold body temperature, obesity, the patient's skin color, or underlying medical conditions.

### SUMMARY OF THE INVENTION

In one aspect, the invention relates to a warming garment for inducing vasodilation comprising an inner lining, an outer lining disposed on the inner lining forming an annular space therebetween, at least one exothermic composition, the at least one exothermic composition disposed within the annular space, and an oxygen port, the oxygen port in fluid communication with the at least one exothermic composition.

In another aspect, the invention relates to a warming garment for inducing vasodilation comprising an inner lining, an outer lining disposed on the inner lining forming an annular space therebetween, at least one exothermic composition, the at least one exothermic composition disposed within the annular space, and an oxygen impermeable package encapsulating the warming garment containing the exothermic composition.

In another aspect, the invention relates to a method of inducing vasodilation for venipuncture, the method comprising placing a tubular warming garment containing an exothermic composition on a subject's limb, exposing the exothermic composition to a reactant to produce heat, inducing vasodilation in the subject's limb, and accessing a vein for venipuncture.

In other aspects, the invention relates to a warming garment wherein the inner lining and the outer lining forms a tubular shape; the warming garment is disposable; the warming garment has at least one open end; the warming garment further comprising an oxygen impermeable package, the oxygen impermeable package encapsulating the warming garment; wherein the temperature of the exothermic composition ranges from 110 to about 120 degrees Fahrenheit; wherein the outer lining is oxygen permeable; the warming garment further comprising a temperature gauge, the temperature gauge fastened to the outer lining; and a warming garment wherein the at least one exothermic composition is sealed inside the annular space.

In another aspect, the invention relates to a method of inducing vasodilation for venipuncture, the method further comprising removing the warming garment from an oxygen impermeable package, connecting the warming garment to an oxygen supply, reading a temperature from a temperature gauge, removing the warming garment from the subject's limb, locating the vein, and puncturing the vein.

### BRIEF DESCRIPTION OF THE DRAWINGS

FIG. 1 provides a perspective view of a warming garment on a subject's limb.

FIGS. 2(a) and 2(b) provide cross-sectional views of different embodiments of a warming garment.

FIG. 3(a) provides a perspective view of a warming garment with exothermic compositions distributed circumferentially around the garment.

FIG. 3(b) provides a perspective view of a warming garment with an array dispersion pattern of the exothermic composition around the garment.

FIG. 4 illustrates a flowchart diagramming the methodology of inducing vasodilation for venipuncture.

FIG. 5 illustrates a flowchart diagramming the methodology of making a warming garment for inducing vasodilation for venipuncture.

### DETAILED DESCRIPTION

Applying heat to an area where venipuncture or intravenous catheter insertion is desired can facilitate and induce vasodilation. Traditional methods or devices include hot towels, however, more sophisticated apparatuses may implement electric heating elements and suction devices. These methods and or devices, have proved to be cumbersome, bulky, and time consuming.

In one set of embodiments, a warming garment is capable of assisting medical practitioners to achieve vasodilation in patients in preparation for venipuncture or intravenous catheter insertions within a matter of minutes. They further provide patient comfort and heat to the applied area immediately upon exposure to oxygen. Once the procedure is completed, components of this set of embodiments allow for easy disposal without any worry of cleaning or disinfecting for repeated use. Furthermore, this set of embodiments provide for efficient packaging and transportation of the apparatus and allow medical practitioners to perform effective venipuncture procedures with adult and young patients.

As used herein, the term “exothermic composition” means a packaged mixture of components that when exposed to oxygen, initiates a chemical reaction that gives off heat.

As used herein, the term “venipuncture” means a procedure involving a surgical puncture of a vein, especially for the withdrawal of blood, intravenous access, and or administration of medications.

As used herein, the term “vasodilation” means a process consisting of the dilation of a blood vessel, as by the action of drugs or heat.

As used herein, the term “annular space” means the area of space formed between the inner and out linings of one aspect of the invention.

As used herein, the term “oxygen impermeable fabric” means a fabric that precludes the penetration or entrance of oxygen.

All definitions, as defined and used herein, should be understood to control over dictionary definitions, definitions in documents incorporated by reference, and/or ordinary meanings of the defined terms.

In one embodiment, the warming garment may be shaped to conform to a subject's limb in a fashion that is not excessively bulky or uncomfortable to wear. Specifically, the warming garment may be shaped into a substantially tubular configuration having one or two open ends and one or zero closed ends. The warming garment may also have an inner surface and an outer surface, where the inner and outer surface forms one or more annular spaces in between. Additionally, in one end of the embodiment, the warming garment may have a elastic component that can secure the garment on a subject's limb such that it's position does not shift while in use, however, other materials and forms may be used to secure the warming garment in place.

In another embodiment, the warming garment may be made out of any material that can safely contact a subject's limb as well as conduct heat to that limb. The material may be made up of two layers of fabric, an outer lining and an inner lining. The fabric of the outer and inner linings can be flexible, woven, nonwoven, jersey, or made of an extruded material. In particular, the inner lining can be compatible with a subject's skin and capable of transferring heat while at the same time, acting as a shield to protect the surface of a subject's limb from excessive heat. The inner lining may also be oxygen permeable or impermeable. The outer lining can be made of the same or different material as the inner lining. The outer lining may also be oxygen permeable as to allow oxygen to flow through the lining and into the annular space formed between the inner and outer surfaces of the warming garment. Furthermore, the warming garment may have a flap that bypasses the outer lining and inner lining of the garment which can be opened for direct access to a vein during venipuncture or catheter insertion procedures rather than having to remove the entire garment.

In yet another embodiment, the warming garment may include a composition that can provide heat to a subject to induce vasodilation in preparation for venipuncture. The composition can produce heat upon exposure to a reactant. There is no need of electricity, hot water, or the like. The composition may be an exothermic composition that can be oxidized to produce heat. In one embodiment, the exothermic composition can react with oxygen to produce heat. In another embodiment, the exothermic composition can include a metal or metals, such as iron powder, zinc powder, aluminum powder or magnesium powder or pulverized alloy of two or more of these metals free from generating odor but most preferred is iron powder with its safety, ease of handling, cost, storage property and stability. In an alternate embodiment, the exothermic composition may be of various forms. Such forms may include but are not limited to a solid, gel, granular, monolithic, or powder form. Examples of iron-containing exothermic compositions include those described in Yamashita et al., U.S. Pat. No. 3,976,049, Yamaguchi et al., U.S. Pat. No. 4,225,157; and Usui, U.S. Pat. No. 5,046,479, the entire contents of which are incorporated herein by reference.

The composition may consist of various compounds such as iron sulfate powder, salt, water, an absorbent material, and activated carbon, but may also include other compounds. The absorbent material helps retain moisture so a reaction can occur at a quick rate and thus produce heat quickly and the activated charcoal allows for even distribution of heat once the compounds are activated. The absorbent material may be vermiculite, pulverized wood, or a superabsorbent material such as polyacrylate. It may contain other fillers not necessarily reactive to oxygen.

To secure the contents of the exothermic composition, the aforementioned compounds may be packaged into an annular space formed between the outer lining and inner lining. Individual packets containing exothermic composition may be made of any oxygen permeable material such as paper, fabric, and permeable polymers. Such packets may be obtained from GRABBER®, Inc. (Michigan).

To prevent the composition from reacting, the warming garment containing the exothermic composition may be packaged in an oxygen impermeable package that is devoid of oxygen. In one embodiment, the oxygen impermeable package may be flexible and made of a polymeric material. The package can be composed of rigid, semi-rigid or flexible material or a combination thereof. The warming garment may be stored in a thermoplastic or a thermosetting polymer such as, but not limited to, high-density or low-density polyethylene, polystyrene, polypropylene, polyvinyl chloride, polyester, polyethylene terephthalate, polyvinylidene chloride, or polyamide. Furthermore, the oxygen impermeable package may be clear such that the contents contained within can be viewable. In another embodiment, the oxygen impermeable package may be opaque, transparent, or translucent. The oxygen impermeable package may also be sealed. For instance, in one embodiment, the oxygen impermeable package can be sealed using a number of different methods including a zipper seal, adhesive, heat seal, ultrasonic weld, vacuum seal, and slide seal.

In another embodiment, the warming garment may provide heat to a subject's limb to induce vasodilation in preparation for venipuncture in a simple, easy, and quick manner. Particularly, once the warming garment is removed from an oxygen impermeable package that is devoid of oxygen, oxygen is able to immediately flow through the oxygen permeable outer lining and into the exothermic composition contained between the outer and inner lining, and thus initiating an exothermic reaction. Consequently, the warming garment begins to increase in temperature and thereby induces vasodilation to a limb that is in contact with the warming garment. The heat produced by the exothermic composition may range from about 110 to 120 degrees Fahrenheit, however, this temperature range may be broader depending on such factors as the components used, environmental factors, amount of exothermic composition, and the amount of oxygen supplied. A temperature ranging from about 90 to 110 degrees Fahrenheit may be enough to induce vasodilation; however, this temperature range may be broader in some embodiments. As heat is produced it can be distributed directly to the surface of a subject's limb where vasodilation and venipuncture procedures are desired.

In another embodiment, the outer lining of the warming garment may be removable to increase exposure of oxygen to the exothermic composition thereby increasing the rate of reaction of the exothermic composition and producing more heat. In an alternate embodiment, exposure to oxygen may be increased by turning the warming garment inside out such that the inner lining may be directly exposed to oxygen if it has not yet been removed.

In another embodiment, if heat is desired quickly, an oxygen supply may be attached to an oxygen port located on the warming garment. The port may provide fluid communication between an oxygen source and the exothermic composition. The garment may also include one or more exhaust vents to provide an exit for oxygen or other gases that may be formed. The port may include a fastener for fastening an oxygen line to the port, where such a fastener may be a LUER-LOK® type fitting. Oxygen flow can be increased to accelerate an increase in temperature and decreased to reduce or stop the increase in temperature. When a desired temperature is reached, the warming garment can easily be withdrawn by loosening the elastic component of the garment and sliding the garment off of a subject's limb. After the warming garment is removed, a vein can be accessed for venipuncture or intravenous catheter insertion. If a vein cannot be accessed, the warming garment may be placed back on the subject's limb for additional heating.

When a used warming garment is no longer needed, disposal can be safe and convenient. Since the exothermic composition can be disposed of anywhere, cleanup can be simple, efficient, and quick. The warming garment will not only improve venipuncture or catheter insertion practices, but it will also assist in providing a more sanitary and safe environment for facilities and personnel that make use of such garments.

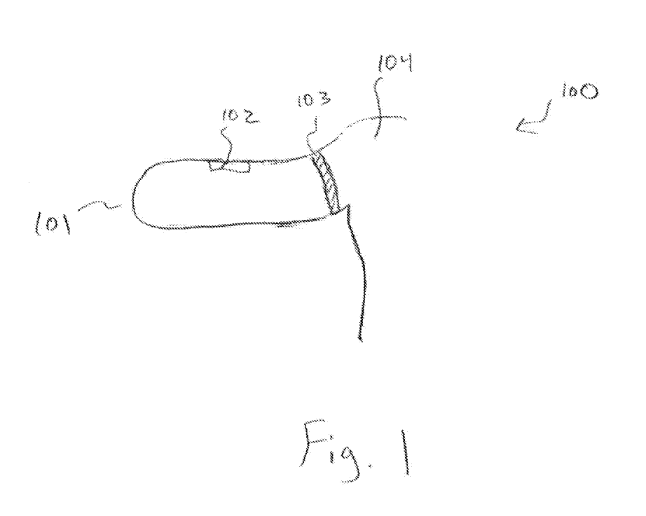

FIG. 1 illustrates one embodiment 100 of a warming garment 101 that is capable of being worn by a subject 104. The warming garment 101 may have a substantially tubular shape as to be capable of fitting a subject's limb. The warming garment 101 may also have a temperature gauge 102 that allows a practitioner or user of the garment to monitor heat applied to a subject and prevent the occurrence of excessive heat that may harm the subject. Although the amount of heat applied to the surface of a subject's limb may vary, a temperature of at most 120 degrees Fahrenheit is preferred. To assure that the warming garment 101 is securely attached to the subject's limb, an elastic component 103 may be used to tighten the garment around the subject's limb such that heat may be applied to the desired area to effectively induce vasodilation.

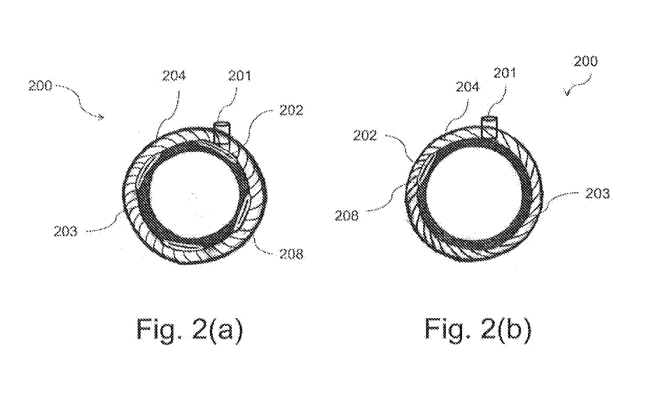

As illustrated in FIG. 2(a) and FIG. 2(b) of other embodiments, the warming garment 101 may have an inner lining 203 and an outer lining 204 that forms a substantially tubular shape. The inner lining 203 and/or outer lining 204 may be moisture permeable. The inner lining 203 and outer lining 204, which may be fabric made of flexible, woven, nonwoven, or extruded material, may form an annular space 208 that is between the two layers. The inner lining 203 may be oxygen impermeable and the outer lining 204 may be oxygen permeable. Furthermore, the inner lining 203 may also act as a conductor of heat while at the same time, provide sufficient shielding and protection of the surface of a subject's limb from uncomfortable heat.

FIG. 2(a) and FIG. 2(b) also illustrate different embodiments of the warming garment 101 that are capable of retaining a composition that can provide heat to a subject 104. The composition that can provide heat to a subject 104 may be an exothermic composition 202. The arrangement of the exothermic composition 202 and the amount or number of exothermic composition 202 may vary. For instance, FIG. 2(a) illustrates an embodiment of the warming garment 101 that is capable of providing heat to a limb with a plurality of annular spaces 208 in which exothermic compositions 202 may be disposed. Additionally, FIG. 2(b) illustrates yet another embodiment of the warming garment 101 that is capable of providing heat to a limb wherein a single exothermic composition 202 is disposed in a single annular space 208 between the inner lining 203 and outer lining 204.

The exothermic composition 202 can produce heat upon exposure to a reactant. Heat may be provided without the use of electricity, water, or any additional source. Instead, the exothermic composition 202 may emit heat upon the exposure to oxygen as oxygen permeates through the outer lining 204 of the warming garment 101. The exothermic composition 202 may also react upon exposure to the atmosphere. The exothermic composition 202 may include a combination of materials such as, but not limited to, iron sulfate, sodium chloride, water, an absorbent material, and activated carbon.

FIGS. 2(a) and 2(b) provide cross-sectional views of various embodiments showing a warming garment 200 that is capable of receiving a reactant that may accelerate the emission of heat. Although oxygen may permeate through the outer lining 204 and initiate an exothermic reaction, the emission of heat can be accelerated with a controlled flow of oxygen. The supply of oxygen may be provided through an oxygen supply by such means as from a hospital or clinical oxygen unit. The flow of oxygen from the oxygen supply may be attached to the warming garment 200 by an oxygen port 201. The oxygen port 201 may be positioned on the outer lining 204, which allows for efficient control of the flow of oxygen directly to the exothermic composition 202 to accelerate or decelerate the rate at which heat is generated. In an alternate embodiment, the supply of oxygen through the oxygen port 301 may be the only means of activating the exothermic composition 202 where the inner lining 203 and outer lining 204 are impermeable to oxygen. Oxygen may be supplied at a rate of, for example, greater than or equal to one liter of oxygen per minute.

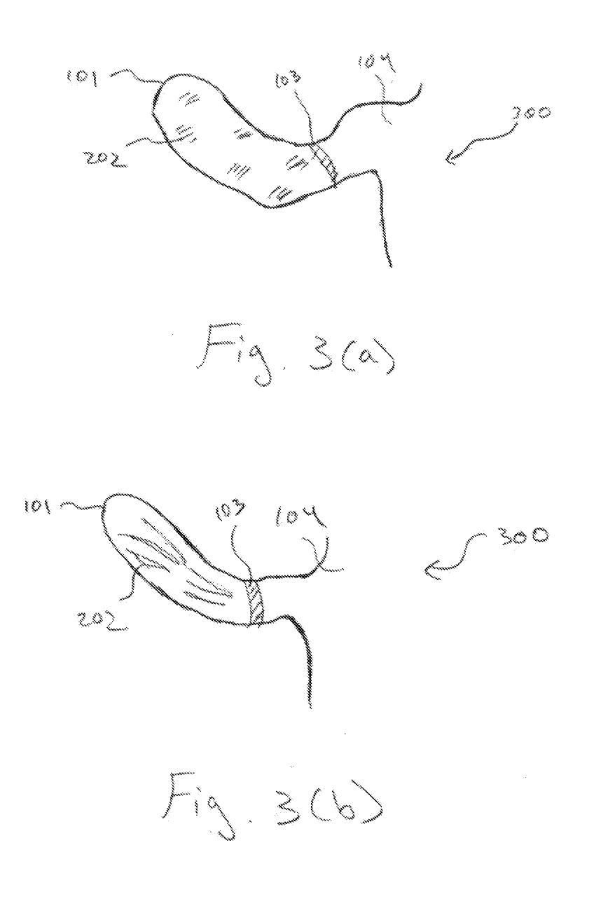

Alternatively, in another embodiment, FIG. 3(a) illustrates a warming garment 101 wherein the exothermic composition 202 is circumferentially distributed throughout a substantial portion of the warming garment 101. In a different embodiment, FIG. 3(b) illustrates a warming garment 101 wherein the exothermic composition 202 is arranged in an array-like fashion that circumferentially surrounds a substantial portion of the warming garment 101.

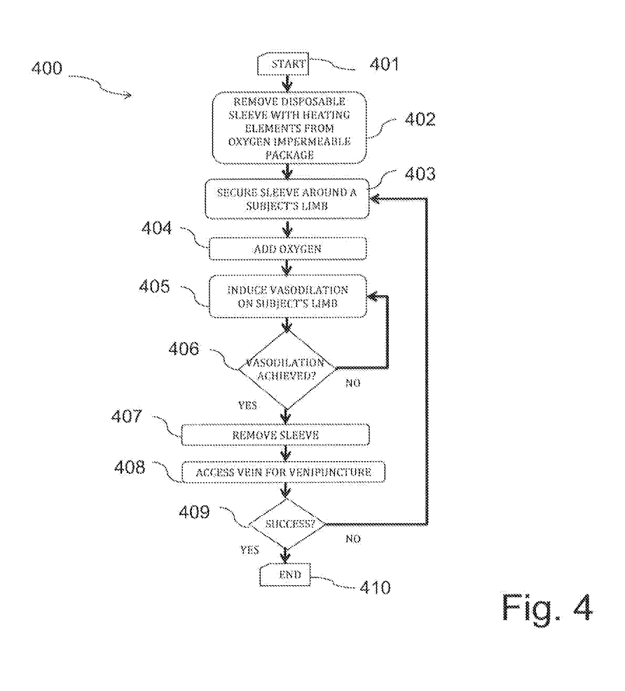

FIG. 4 illustrates a flow diagram 400 of a method of using the present invention. The warming garment 101 can be used in a variety of methods. The flow diagram 400 begins at step 401. At step 402, a warming garment 101 is removed from an oxygen impermeable package. At step 403, the warming garment 101 is placed on a subject's limb. At step 404, oxygen supplied to the warming garment 101. At step 405, vasodilation is induced on the subject's limb. At step 406, if vasodilation is achieved, then at step 407, the warming garment is removed. At step 408, a vein is accessed for venipuncture. At step 409, if a vein was successfully accessed, then at 410, the flow diagram 400 ends. If vasodilation is not achieved at step 405, then step 404 is repeated until vasodilation is achieved.

In another embodiment, the method of the flow diagram 400 further includes connecting the warming garment 101 to an oxygen supply. In an alternate embodiment, the method of the flow diagram 400 also includes reading a temperature from a temperature gauge 301. In still another embodiment, the method of the flow diagram 400 includes removing the warming garment 101 from the subject's limb. In yet another embodiment, the method of the flow diagram 400 includes puncturing the vein.

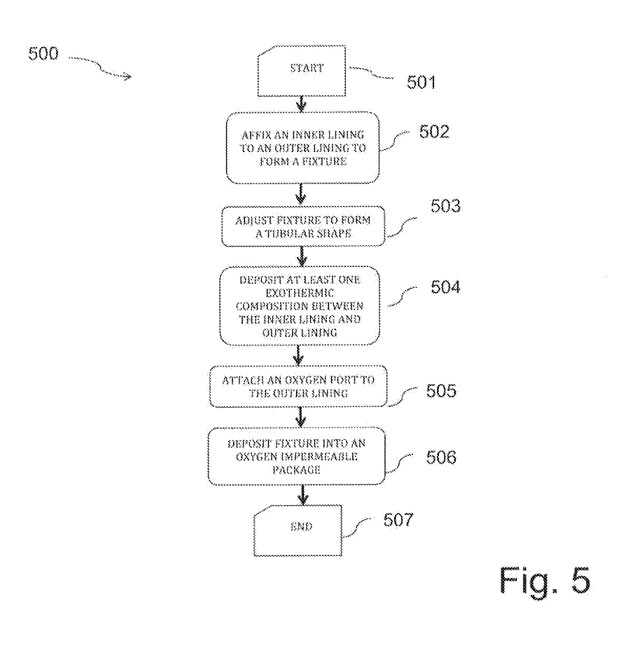

FIG. 5 illustrates a flow diagram 500 describing a method of making a warming garment. The warming garment 101 may be made by a variety of methods. The flow diagram 500 begins at step 501. At step 502, an inner lining 203 is affixed to an outer lining 204 to form a fixture. At step 503, the fixture is adjusted into a tubular shape. At step 504, at least one exothermic composition 202 is deposited between the inner lining 203 and outer lining 204. At step 505, an oxygen port 301 is attached to the outer lining 204. At step 506, the fixture is deposited into an oxygen impermeable package. At step 507, the flow diagram 500 ends.

It should also be understood that, unless clearly indicated to the contrary, in any methods claimed herein that include more than one act, the order of the acts of the method is not necessarily limited to the order in which the acts of the method are recited. Furthermore, such methods may be performed simultaneously.

Although various embodiments of the present invention have been described herein, the above description is merely illustrative. Further modification of the claimed invention herein disclosed will occur to those skilled in the respective arts and all such modifications are deemed to be within the scope of the invention as defined by the appended claims.

In the claims as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A warming garment for inducing vasodilation comprising:
an inner lining;
an outer lining disposed on the inner lining forming an annular space therebetween;
at least one exothermic composition, the at least one exothermic composition disposed within the annular space; and
an oxygen port, the oxygen port in fluid communication with the at least one exothermic composition.

2. The warming garment of claim 1 wherein the inner lining and the outer lining form a tubular shape.

3. The warming garment of claim 1 wherein the garment is disposable.

4. The warming garment of claim 1 wherein the garment has at least one open end.

5. The warming garment of claim 1 further comprising an oxygen impermeable package, the oxygen impermeable package encapsulating the warming garment.

6. The warming garment of claim 1 wherein the garment is capable of heating the skin of a subject to a temperature ranging from about 110 to about 120 degrees Fahrenheit.

7. The warming garment of claim 1 wherein the outer lining is oxygen permeable.

8. The warming garment of claim 1 further comprising a temperature gauge, the temperature gauge fastened to the outer lining.

9. The warming garment of claim 1 wherein the at least one exothermic composition is sealed between the inner lining and the outer lining.

10. A warming garment for inducing vasodilation comprising:
an inner lining;
an outer lining disposed on the inner lining forming an annular space therebetween;
at least one exothermic composition, the at least one exothermic composition disposed within the annular space; and
an oxygen impermeable package encapsulating the warming garment containing the exothermic composition.

11. The warming garment of claim 10 further comprising an oxygen port, wherein the oxygen port is in fluid communication with the at least one exothermic composition.

12. The warming garment of claim 10 wherein the inner lining and the outer lining form a tubular shape.

13. The warming garment of claim 10 wherein the garment is disposable.

14. The warming garment of claim 10 wherein the garment has at least one open end.

15. The warming garment of claim 10 wherein the garment is capable of heating the skin of a subject to a temperature ranging from about 110 to about 120 degrees Fahrenheit.

16. The warming garment of claim 10 wherein the outer lining is oxygen permeable.

17. The warming garment of claim 10 further comprising a temperature gauge, the temperature gauge fastened to the outer lining.

18. The warming garment of claim 10 wherein the at least one exothermic composition is sealed between the inner lining and the outer lining.

19. A method of inducing vasodilation for venipuncture, the method comprising:
placing a tubular warming garment containing an exothermic composition on a subject's limb;
exposing the exothermic composition to a reactant to produce heat;
inducing vasodilation in the subject's limb; and
accessing a vein for venipuncture.

20. The method of claim 19 further comprising removing the warming garment from an oxygen impermeable package.

21. The method of claim 19 further comprising connecting the warming garment to an oxygen supply.

22. The method of claim 19 further comprising reading a temperature from a temperature gauge.

23. The method of claim 19 further comprising removing the warming garment from the subject's limb.

24. The method of claim 19 further comprising locating the vein.

25. The method of claim 19 further comprising puncturing the vein.
